(19) United States
(12) Patent Application Publication
(10) Pub. No.: US 2026/0207351 A1
(43) Pub. Date: Jul. 23, 2026

(54) BREAST PROSTHESIS

(71) Applicant: ONEBRA S.R.L., TRENTO (IT)

(72) Inventors: Sofia SANTI, TRENTO (IT); Silvia CHIERA, TRENTO (IT); Danilo TOMASONI, TRENTO (IT); Giulia FAORO, TRENTO (IT); Ciro MALACARNE, TRENTO (IT)

(21) Appl. No.: 19/139,154

(22) PCT Filed: Dec. 14, 2023
(86) PCT No.: PCT/IB2023/062706

§ 371 Date: Jun. 13, 2025

(30) Foreign Application Priority Data
Dec. 15, 2022 (IT) 102022000025716

(51) Int. Cl. A61F 2/52

(52) U.S. Cl. CPC A61F 2/52

(57) ABSTRACT

The present invention relates to a breast prosthesis. More specifically, it is a breast prosthesis for external use. The breast prosthesis (10) comprises a lattice structure (12) made from a network of filaments (14).

The present invention relates to a breast prosthesis. More specifically, it is a breast prosthesis for external use.

As is well known, following an operation to remove the breast or a portion of it, prosthesis may be used to give the breast the appropriate volume.

Such prostheses can be placed internally or externally on the body of the woman who has undergone such removal.

On the one hand, internal prostheses are comfortable, but on the other hand they cannot be easily replaced or adapted to the needs of the woman wearing them. In addition, internal prostheses can cause infections and pain (e.g., one must maintain a supine position to avoid pain while sleeping) and they can shift years later.

In order to overcome this limitation, in most women who have undergone mastectomy surgery, external prosthesis are used, which are usually made of silicone or gel contained in a suitably shaped package.

These prostheses are arranged in a brassiere with pockets for stable and firm containment of the prostheses.

One of the major problems with external prostheses made according to the known technique lies in their noticeable weight, which makes them uncomfortable to use.

A further problem with external prostheses is their almost complete lack of breathability.

Another issue with known external prostheses is the long lead times.

A further problem with the prosthesis currently on the market lies in their dimensions, which are standard and therefore cannot accommodate all breast conformations.

The aim of the present invention is to provide an external breast prosthesis that overcomes, at least partially, the problems of the known technique.

A further aim of the invention is to offer an external breast prosthesis that is comfortable in terms of fit.

Another purpose of the invention is to provide an external breast prosthesis that is breathable so that the wearer is comfortable.

A further aim of the invention is to offer an external breast prosthesis that has an optimal weight according to the build of the woman who has to wear it.

Another purpose of the invention is to provide an external breast prosthesis that can be made quickly and easily.

These and other purposes are achieved by a breast prosthesis that is suitable for external use, e.g. inside a bra pocket, characterised by the fact that it comprises a lattice structure made from a network of filaments.

Specifically, it is a customised external prosthesis, essentially a customised cup, made by means of three-dimensional printing with a lattice structure.

Thanks to this conformation, the prosthesis is comfortable, light and breathable.

Moreover, thanks to the three-dimensional printing technique used, the conformation of the prosthesis can be easily adapted to the physiognomy of the breast.

Advantageously, the breast prosthesis according to the invention may comprise a first sheet arranged on the front portion of the lattice structure, so as to make the lattice structure itself stable.

In addition, the breast prosthesis may include a second lamina, placed on the rear portion of the lattice structure, to further improve the stability of the lattice structure.

Advantageously, the lattice structure can be made of thermoplastic polyurethane (TPU), so it can be easily obtained, e.g. by 3D moulding.

The use of such a material also allows the cost and time of the prosthesis itself to be safeguarded.

Advantageously, the lattice can have a hardness of between 90 and 95 Shore A so that the lattice structure is, on the one hand, sufficiently rigid to give stability to the entire prosthesis and, on the other hand, still elastic to ensure comfort and a good fit.

In addition, the filament and/or foils can be between 0.8 mm and 2.5 mm thick, so that the lattice structure and the entire prosthesis is breathable and sufficiently light.

Also for this purpose, the lattice structure can have a volume of solid relative to void of between 6% and 15%.

Advantageously, the breast prosthesis according to the invention may also comprise an injected material within the lattice structure, so that the weight of the entire prosthesis can be adjusted according to the needs of the wearer.

In addition, for optimal weight, the injected material can have a density of 1.06 g/mL or more.

In order to ensure the breathability of the prosthesis, the injected material may be porous.

Further features and details can be better understood from the description below, which is given as a non-limiting example, as well as from the attached drawing board, where:

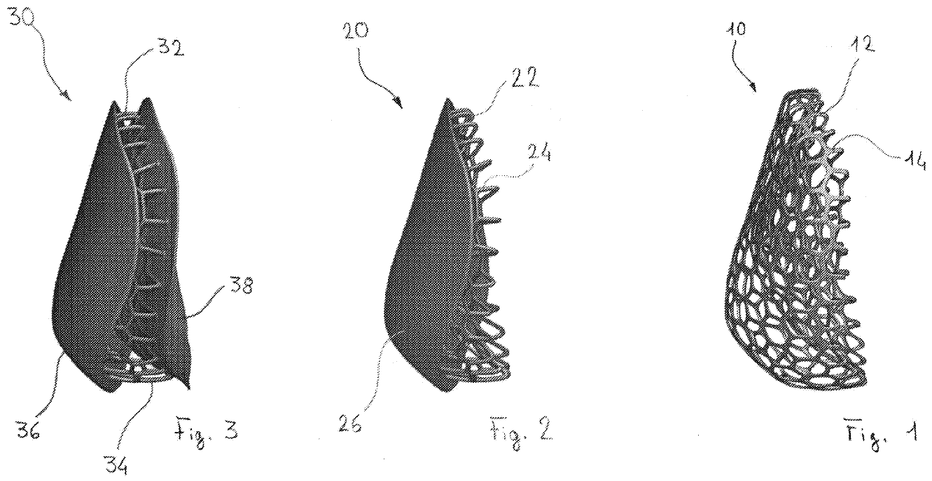

FIG. 1 is a three-dimensional schematic view of an external breast prosthesis according to the invention;

FIG. 2 is a three-dimensional schematic view of an external breast prosthesis according to an early variant of the invention;

FIG. 3 is a three-dimensional schematic view of an external breast prosthesis according to a second variant of the invention.

With reference to FIGS. 1, 10 identifies a breast prosthesis for external use to be placed in a suitably constructed pocket in a bra to prevent its movement.

The breast prosthesis 10 comprises a lattice structure 12 made from a lattice of filaments 14.

According to a first variant of the invention, illustrated in FIG. 2, a breast prosthesis 20 for external use comprises a lattice structure 22 made from a lattice of filaments 24.

The breast prosthesis 20 comprises a first lamina 26 placed on the front portion of the lattice structure 22.

The first lamina 26 has a conformation similar to that of the surface of a woman's natural breast.

According to a second variant of the invention, illustrated in FIG. 3, a breast prosthesis 30 for external use comprises a lattice structure 32 made from a lattice of filaments 34.

The breast prosthesis 30 comprises a first lamina 36 and a second lamina 38, arranged on the front and rear portions of the lattice structure 32, respectively.

The lattice structure 12, 22, 32 allows for appropriate softness and lightness. In particular, lattice structure 12, 22, 32 can be made of thermoplastic polyurethane (TPU) and thus have a hardness between 90 and 95 Shore A, preferably 93 Shore A.

Filament 14, 24, 34, as well as foils 26, 36, 38 can be between 0.8 and 2.5 mm thick.

The lattice structure 12, 22, 32 can have a volume of solid relative to void of between 6% and 15%.

This range mainly guarantees lightness and breathability, and is independent of the material used.

In all three embodiments of the invention illustrated above, the lattice structure includes knots; the set distance for the generation of latex knots is approximately 10 mm, but this can of course vary depending on the needs of the embodiment.

Foils 26, 36, 38 have a thickness similar to that of the filament, as illustrated above.

According to a variant of the invention, a prosthesis similar to the one described above may comprise a material injected into the lattice structure.

The injected material has a density of more than 1.06 g/mL (TPU) and has a porous structure to ensure breathability.

The lattice structure thus becomes the supporting structure of the prosthesis.

According to another variant of the invention, a prosthesis possibly comprising injected material is provided with a 3D-printed TPE (thermoplastic elastomer) covering of Shore 00 to ensure a natural softness of the cup and maintain the requirement of breathability.

According to a further variant of the invention, a prosthesis similar to the one described above can comprise a lattice structure with voronoids that are wider away from the edges and narrower near the edges.

This allows for greater stability near the edges and greater lightness away from the edges.

The application mode of the breast prosthesis according to the invention is as follows.

If there has been a total removal of an udder, pellets including TPE are inserted into the lattice structure, with high breathability due to the characteristics of each piece of pellet, which has a size of approximately 5×5 mm. The final weight must correspond to 18-20 times the weight of the empty lattice structure.

In particular, the TPE material used is a thermoplastic elastomer of Shore 00.

If there has been a quadrantectomy or we are in the presence of an prosthesis that creates asymmetry in relation to the other breast, wadding must be inserted into the lattice structure until the final weight of the cup is approximately 1.5 to 1.7 times the weight of the empty lattice structure.

Further variants and implementation methods other than those illustrated above are possible and are to be considered within the scope of protection defined by the attached claims.

For example, the conformation of lattice structures 12, 22, 32 may differ from those shown in the attached figures.

1. Breast prosthesis suitable for use externally, e.g. inside a bra pocket, characterised by the fact of comprising a lattice structure made from a network of filaments.

2. Breast prosthesis according to claim 1, wherein a first lamina arranged on the front portion of the reticular structure is included.

3. Breast prosthesis according to one claim 2, wherein a second lamina arranged on the rear portion of the lattice structure (32), is included.

4. Breast prosthesis according to claim 1, wherein the lattice structure is made of thermoplastic polyurethane (TPU).

5. Breast prosthesis according to claim 4, wherein the lattice structure has a hardness of between 90 and 95 Shore A.

6. Breast prostheses according to claim 1, wherein the filament and/or foils are between 0.8 mm and 2.5 mm thick.

7. Breast prostheses according to claim 1, wherein the lattice structure has a volume of the solid relative to the void of between 6% and 15%.

8. Breast prosthesis according to claim 1, wherein an injected material is included within the lattice structure.

9. Breast prosthesis according to claim 8, wherein the injected material has a density of 1.06 g/mL or more.

10. Breast prosthesis according to claim 8, wherein the injected material is porous.

11. Breast prosthesis according to claim 1, wherein the lattice structure has a voronoi conformation.

12. Breast prosthesis according to claim 11, wherein the lattice structure has a voronoid conformation that is wider away from the edges and narrower near the edges.

13. Breast prosthesis according to claim 1, wherein a filler material is placed in the lattice structure.

14. Breast prosthesis according to claim 13, wherein the filler material comprises pellets and/or wadding.

15. Breast prosthesis according to claim 1, wherein a covering which covers the lattice structure, is included.

16. Breast prosthesis according to claim 15, wherein the covering is made of TPE.

17. Breast prosthesis according to claim 16, wherein the covering is made of 3D-printed Shore 00 TPE.

18. Breast prosthesis according to claim 1, wherein the lattice structure has a hardness of between 90 and 95 Shore A.

19. Breast prosthesis according to claim 1, wherein the injected material is porous.

20. Breast prosthesis according to claim 11, wherein a filler material is placed in the lattice structure.
